(19) United States
(12) Patent Application Publication
(10) Pub. No.: US 2022/0165410 A1
(43) Pub. Date: May 26, 2022

(54) REMOTE COMMUNICATION WITH A MEDICAL TECHNOLOGY FACILITY WITH THE AID OF A DIGITAL TWIN

(71) Applicant: Siemens Healthcare GmbH, Erlangen (DE)

(72) Inventors: Verena SCHMIDT, Erbendorf (DE); Joerg HOFMANN, Forchheim (DE); Stefan FRIEDRICH, Bayreuth (DE)

(73) Assignee: Siemens Healthcare GmbH, Erlangen (DE)

(21) Appl. No.: 17/522,035

(22) Filed: Nov. 9, 2021

(30) Foreign Application Priority Data
Nov. 20, 2020 (DE) 10 2020 214 654.3

(51) Int. Cl. G16H 40/67; G16H 40/40

(52) U.S. Cl. CPC G16H 40/67; G16H 40/40

(57) ABSTRACT

A remote communication facility is described. The remote control facility of an embodiment includes a data capture unit for capturing state data and machine data from a medical technology facility; an evaluating unit for generating a digital twin based upon the state data and machine data; a display unit for displaying the digital twin and a control unit for controlling the medical technology facility based upon the displayed data of the digital twin. In addition, an operation and treatment system is described. A remote communication method is also described.

### PRIORITY STATEMENT

The present application hereby claims priority under 35 U.S.C. § 119 to German patent application number DE 102020214654.3 filed Nov. 20, 2020, the entire contents of which are hereby incorporated herein by reference.

### FIELD

Example embodiments of the invention generally relate to a remote communication facility; an operation and treatment system; and/or a remote communication method.

### BACKGROUND

Modern medical technology devices are indispensable for the examination, treatment, care and monitoring of patients.

For example, medical technology devices are used for imaging methods which can be utilized for visualizing an imaged examination object being mapped and additionally for further uses. Examples of modern medical imaging technology methods are computed tomography, magnetic resonance tomography, the use of what are known as PET scanners and X-ray imaging with C-arm systems.

In particular, in medical technology facilities that operate with X-rays, it is necessary to provide protective measures for the medical personnel in order to keep the exposure as low as possible. One possibility therein lies in the remote control of such devices. For example, conventionally, a person sits in an adjoining room separate from the medical imaging technology facility, with a viewing window and sight of the patient, and starts an imaging process from the adjoining room.

However, it is desirable that the medical imaging technology facility could also be controlled and monitored without a direct view of the patient or even somewhere at another location in the world. Such an approach would be particularly advantageous due to the worldwide shortage of qualified specialist personnel, since then patients could be examined worldwide even where there is no relevant specialist personnel.

If it is desired to monitor the progress of the imaging with cameras, then arranged in the actual examining room in which the medical imaging technology facility is situated with the patient to be examined is a plurality of cameras with which image data of the medical technology facility is captured and, for example, transmitted via the hospital network to a terminal in the adjoining room in which the medical personnel are situated. However, the problem exists that objects can block the field of view of the camera. The clinical personnel therefore has a poorer overview of the situation than directly in reality on site in the examining room. In addition, a plurality of camera images must be viewed in order to obtain a spatial understanding of the situation. The camera images are displayed to an operating person on a plurality of screens in parallel, but must be mentally assembled by the operating person. Specifically, the operating person in the control room receives medical settings of the medical technology facility displayed via an operating panel, for example, the X-ray dose or the body program, but he receives no information regarding the current maintenance condition of the device and/or the medical technology facility and no machine data.

### SUMMARY

The inventors have discovered that the exact operational state of the medical technology device is discernible only with difficulty or, according to the circumstances, not at all. In addition, the inventors have discovered that the additional data for determining a current operational state of a medical technology device must then be communicated and displayed separately.

At least one embodiment of the present invention provides a remote control system of a medical technology device with improved monitoring and control possibilities, regardless of the location of the medical personnel taking action.

Embodiments according to the invention are directed to a remote communication facility, an operation and treatment system and a remote communication method.

The remote communication facility according to at least one embodiment of the invention has a data capture unit for capturing state data from a medical technology facility. Status data is all data which describes the current technical state of a medical technology facility. This state data can comprise, for example, operational parameters with which the medical technology facility is currently operated. The operational parameters comprise, in the case of an X-ray imaging facility, for example, the X-ray voltage or the organ program. The state data can also comprise machine data. The machine data comprises, for example, the joint position of the device and/or the medical technology facility or the sensor data. The internal communication and control in the medical technology facility is brought about via machine data. The machine data is the data that is used internally in the medical technology facility for controlling individual function units or generally for the communication of individual components with one another. In addition to operational parameters, the state data can thus also comprise machine data that must still be evaluated in order to be able to determine the current state of a medical technology facility.

The operation and treatment system according to at least one embodiment of the invention has an operation and/or treatment room with a medical technology facility or a plurality of medical technology facilities and a control room spatially separated from the operation and/or treatment room with a remote communication facility according to the invention. The control room can therein suffice without visual contact with the medical technology facility and the patient.

In the remote communication method according to the invention, state data based, for example, upon machine data, or operational parameters of a medical technology facility are captured. The state data comprises not only the operational parameters which are received from the medical technology facility, but also product data from the manufacturer, for example, to be able to assign the operational parameters correctly to a 3D visualization. The state data can be transferred to a remote control facility, but it can also be pre-processed in a so-called edge pre-processing directly in or at the medical technology facility. Based upon the state data, a digital twin is generated. The digital twin can be generated either, as already mentioned, by a computer unit spatially assigned to the medical technology facility and then transferred to the control room or it can first be generated in the control room and then displayed spatially remotely from the medical technology facility. In addition, from the control room, a remote communication with the medical technology facility takes place. This communication can comprise a spatially remote controlling of the medical technology facility based upon the displayed data of the digital twin. The remote communication can, however, also comprise a device monitoring for a servicing or a monitoring of a patient who is located in the admission region of the medical technology facility. For example, an intervention on the patient can be followed remotely by students or scientists based upon the digital twin in order to learn new methods and procedures. The remote communication method according to at least one embodiment of the invention includes the advantages of the remote communication facility according to at least one embodiment of the invention.

A realization largely through software has the advantage that conventionally used remote control facilities, possibly with retrofitting of necessary hardware, for example, a data capture unit, can easily be configured with a software update to operate in the manner according to at least one embodiment of the invention. In this respect, at least one embodiment of the invention is also directed to a corresponding computer program product with a computer program which is loadable directly into a storage apparatus of a remote communication facility and comprises program portions in order to carry out all the steps of the method according to at least one embodiment of the invention when the computer program is executed in the remote communication facility.

At least one embodiment of the invention is further directed to a computer program stored on a computer-readable medium. For transport to the storage facility of a data processing facility and/or for storage at the data processing facility, a computer-readable medium, for example, a memory stick, a hard disk drive or another transportable or firmly installed data carrier can be used on which the program portions of the computer program which are configured to be read in and executed by a data processing facility, for example, a computer unit are stored. For this purpose, the computer unit can have one or more cooperating microprocessors or the like, for example. For example, a cloud system or a database can come into consideration as a storage facility. The transfer of the program can thus also take place within a data network and/or the Internet.

At least one embodiment of the invention is further directed to a remote communication facility, comprising:

a data capture unit to capture state data from a medical technology facility;

an evaluating unit to generate a digital twin based upon the state data;

a display unit to display the digital twin; and

a communication unit to communicate with the medical technology facility based upon displayed data of the digital twin.

At least one embodiment of the invention is further directed to an operation and treatment system, comprising:

at least one of an operation and treatment room including at least one medical technology facility; and

a control room, spatially separated from the at least one of operation and treatment room, including the remote communication facility of an embodiment.

At least one embodiment of the invention is further directed to a remote communication method, comprising:

capturing state data including at least one of machine data and operational parameters of a medical technology facility;

generating a digital twin based upon the state data;

spatially remotely displaying the digital twin; and

spatially remotely communicating with the medical technology facility based upon displayed data of the digital twin.

At least one embodiment of the invention is further directed to a non-transitory computer program product storing a computer program, directly loadable into a storage facility of a data processing facility, including program portions to carry out the method of an embodiment when the computer program is executed in the data processing facility.

At least one embodiment of the invention is further directed to a non-transitory computer-readable medium storing program portions, readable in and executable by a computer unit, to carry out the method of an embodiment when the program portions are executed by the computer unit.

### BRIEF DESCRIPTION OF THE DRAWINGS

The invention will now be described and explained in greater detail making reference to the example embodiments illustrated in the drawings.

In the drawings:

FIG. 1 shows a schematic representation of a medical technology examination system having an X-ray device and a remote communication facility according to an example embodiment of the invention,

FIG. 2 shows a schematic representation of a medical technology examination system having an X-ray device and a remote communication facility according to a second example embodiment of the invention,

FIG. 3 shows a schematic representation of a medical technology examination system having an X-ray device and a remote communication facility according to a third example embodiment of the invention,

FIG. 4 shows a schematic representation of a medical technology examination system having an X-ray device and a remote communication facility according to a fourth example embodiment of the invention,

FIG. 5 shows a flow diagram which illustrates schematically a remote communication method for remote control of a medical technology device according to an example embodiment of the invention,

FIG. 6 shows a flow diagram which illustrates the data transfer processes on a reproduction of a state of a medical technology device on a display of a remote communication facility,

FIG. 7 shows a flow diagram which illustrates the data transfer processes when a medical technology device is controlled via a remote communication facility.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The remote communication facility according to at least one embodiment of the invention has a data capture unit for capturing state data from a medical technology facility. Status data is all data which describes the current technical state of a medical technology facility. This state data can comprise, for example, operational parameters with which the medical technology facility is currently operated. The operational parameters comprise, in the case of an X-ray imaging facility, for example, the X-ray voltage or the organ program. The state data can also comprise machine data. The machine data comprises, for example, the joint position of the device and/or the medical technology facility or the sensor data. The internal communication and control in the medical technology facility is brought about via machine data. The machine data is the data that is used internally in the medical technology facility for controlling individual function units or generally for the communication of individual components with one another. In addition to operational parameters, the state data can thus also comprise machine data that must still be evaluated in order to be able to determine the current state of a medical technology facility.

Furthermore, the remote communication facility according to at least one embodiment of the invention comprises an evaluating unit for generating a digital twin based upon the state data which is based, for example, on machine data and preferably also based upon models of the digitally represented medical technology facility, the state of which is synchronized via the data of the real system. The state data, for example, machine data is preferably stored temporarily in a cloud and can be retrieved by the data capture unit. For example, the raw data, i.e. the machine data, can be sent via the cloud to the evaluating unit from which it is then further processed in order to generate the digital twin. Alternatively, the raw data, that is, the machine data, can be prepared in the medical technology facility such that only the information important for the digital twin is transferred as state data to the evaluating unit. This variant is also referred to as edge processing. The state data can comprise, for example, an item of position information, settings of an organ program or the duration of use so far. The generation of the state data can also take place directly in the medical technology facility or based upon machine data. For example, the determination of the duration of use so far can take place via a counter in the medical technology facility, the count value of which must only be transferred to the evaluating unit via the cloud and/or the evaluating unit. Alternatively, the determination of the duration of use so far can also take place based upon existing machine data. In this case, an evaluation for determining the duration of use so far must take place and this state information is not directly available.

A digital twin is a digital representation of an object from the real world in the digital world. Where reference is made to a real object, this can also comprise a complex system of a plurality of objects which interact with one another dynamically. Apart from remotely controllable objects such as robots, medical technology facilities and the like, such a complex system can also comprise persons who are present in the vicinity of these objects and, where relevant, interact with them. Apart from pure data, a digital twin also comprises a model of the object represented and can also comprise simulations and algorithms which describe or influence properties or behavior of the object or process represented. Therefore, in addition to the digital representation of the real object, a control of the object is also enabled by action upon the digital representation.

The possibility of remote control of the real object is provided. The action on the digital representation thus enables, in particular, the real instance to be controlled accordingly.

For the same object, a plurality of digital twins can exist which are detailed differently according to utilization purpose and have at least partially different features. For example, a digital twin of an X-ray device that is used for the control thereof can also reflect different information than a digital twin which is used for a servicing of the X-ray device.

An operating person can thus communicate indirectly with the real object remotely, purely based upon a reproduction of the digital representation. A digital twin can comprise, for example, a 3D model of the real object. It can, however, also comprise a functional model which maps mechanical, electronic and other properties and performance features of the real object and/or the real twin via model-based embodiments as realistically and completely as possible.

The remote communication facility according to at least one embodiment of the invention also comprises a display unit for displaying the digital twin. Such a display unit can comprise, for example, a screen which represents pictorially the real object and further data linked to the real object, for example, functional data, for example operational parameters, based upon digital data, preferably in real time. So-called virtual reality systems can also be used in place of a conventional screen. Often, so-called dashboards, that is, graphical user interfaces can be used for visualizing important parameters.

The display unit can also comprise a visualization program, for example, Unity, in order to represent the dynamic digital twin graphically and/or the interactive virtually mapped world of the environment of the real object. The remote communication facility according to the invention also comprises a communication unit for communicating with a medical technology facility based upon the displayed data of the digital twin. The communication unit can comprise, for example, a control unit for controlling the medical technology facility based upon the displayed data of the digital twin. By controlling the medical technology facility, an operating person can operate it remotely. The operating person can thereby follow the effect of his control action on the display unit. The control and/or communication with the medical technology facility can also take place via a speech input or gesture control. A speech input could be particularly simple in the case of the remote communication facility according to the invention, since the person carrying it out sits, for example, alone at a PC (possibly with a headset) and the computer can be trained to the operator accordingly.

For example, movements carried out by the medical technology facility are displayed on the screen of the display unit or, based upon the control action, changing operational parameters are displayed, so that the operating person can control and monitor the operation of the medical technology facility remotely. Advantageously, as a result of a combination of a digital model of a medical technology facility with updated operational data and monitoring data, the operating person receives a direct overview of the status of the medical technology facility, which is superior to a pure monitoring via cameras or other sensors, since the digital model then also permits a monitoring of the current state of a medical technology facility, for example, also a view from any desired perspectives onto the medical technology facility if cameras and/or other sensors are blocked by obstacles and/or interfering effects. In addition, details of a scenario that would only confuse the operating person can be hidden, so that the operation of the medical technology facility is simplified for the operating person.

The operation and treatment system according to at least one embodiment of the invention has an operation and/or treatment room with a medical technology facility or a plurality of medical technology facilities and a control room spatially separated from the operation and/or treatment room with a remote communication facility according to the invention. The control room can therein suffice without visual contact with the medical technology facility and the patient.

State data that has been prepared, comprising operational parameters or machine data of medical technology examination devices, treatment devices and operating devices can be captured, for example, via an interface of an electronic control unit of these devices and stored in the cloud and/or an internal network such as, for example, a hospital network. This information can then be implemented in a digital model in real time and displayed visually to the operating personnel spatially remotely in the control room which is equipped, for example, as a remote operating room. The operation and treatment system according to the invention shares the advantages of the remote communication facility according to the invention.

Advantageously, a plurality of medical devices can also be operated simultaneously by one person who has access via the remote communication facility to the control system of a plurality of medical technology facilities. An operation or an X-ray recording can also be carried out remotely by a particularly suitable person without the person having to be on site. In addition, due to the spatial separation of the operating person and the patient, the safety of the medical personnel in the event of a patient having infectious diseases is increased, without an appropriate treatment of the patient having to be dispensed with.

In the case of operations in which a plurality of devices must be used, it is usual that the medical personnel operate a plurality of devices. Therein, during the intervention, the different devices are used sequentially or in combination. The medical personnel themselves determine which device is used, and when.

In laboratories also, different devices are utilized. These function independently and can be finished with the processing at different times. Through the communication of the state to a mobile device, the laboratory staff member knows when, for example, something must be exchanged. He does not need to check the machines continuously, but rather the devices provide information to the user. In this way, unnecessary routes to the checking of the device state are spared and devices are loaded or unloaded at the right time point.

If a plurality of devices from the same manufacturer is used (e.g. in the laboratory) or from compatible manufacturers, the medical personnel can remotely control the sequence of the use of a plurality of devices simultaneously via the digital twin, in that, for example, pre-defined modes of the cooperation are accessed (defined workflows).

In the remote communication method according to at least one embodiment of the invention, state data based, for example, upon machine data, or operational parameters of a medical technology facility are captured. The state data comprises not only the operational parameters which are received from the medical technology facility, but also product data from the manufacturer, for example, to be able to assign the operational parameters correctly to a 3D visualization. The state data can be transferred to a remote control facility, but it can also be pre-processed in a so-called edge pre-processing directly in or at the medical technology facility. Based upon the state data, a digital twin is generated. The digital twin can be generated either, as already mentioned, by a computer unit spatially assigned to the medical technology facility and then transferred to the control room or it can first be generated in the control room and then displayed spatially remotely from the medical technology facility. In addition, from the control room, a remote communication with the medical technology facility takes place. This communication can comprise a spatially remote controlling of the medical technology facility based upon the displayed data of the digital twin. The remote communication can, however, also comprise a device monitoring for a servicing or a monitoring of a patient who is located in the admission region of the medical technology facility. For example, an intervention on the patient can be followed remotely by students or scientists based upon the digital twin in order to learn new methods and procedures. The remote communication method according to at least one embodiment of the invention includes the advantages of the remote communication facility according to at least one embodiment of the invention.

The components of the remote communication facility according to at least one embodiment of the invention can be configured mainly in the form of software components. This relates, in particular, to the evaluating unit and the control unit of the remote communication facility.

Fundamentally however, these components can also, in particular, be realized in part, if particularly rapid calculations are involved, in the form of software-supported hardware, for example, FPGAs or the like. Similarly, the required interfaces can be configured, for example, where only an acceptance of data from other software components is concerned, as software interfaces. However, they can also be configured as interfaces which are constructed as hardware and are controlled by suitable software.

A realization largely through software has the advantage that conventionally used remote control facilities, possibly with retrofitting of necessary hardware, for example, a data capture unit, can easily be configured with a software update to operate in the manner according to at least one embodiment of the invention. In this respect, at least one embodiment of the invention is also directed to a corresponding computer program product with a computer program which is loadable directly into a storage apparatus of a remote communication facility and comprises program portions in order to carry out all the steps of the method according to at least one embodiment of the invention when the computer program is executed in the remote communication facility.

Such a computer program product can comprise, apart from the computer program, additional components, if relevant, such as, for example, documentation and/or additional components including hardware components, for example, hardware keys (dongles, etc.) in order to use the software.

Via a software implementation, the method is executable reproducibly and in a less fault-prone manner on different data processing facilities.

At least one embodiment of the invention is further directed to a computer program stored on a computer-readable medium. For transport to the storage facility of a data processing facility and/or for storage at the data processing facility, a computer-readable medium, for example, a memory stick, a hard disk drive or another transportable or firmly installed data carrier can be used on which the program portions of the computer program which are configured to be read in and executed by a data processing facility, for example, a computer unit are stored. For this purpose, the computer unit can have one or more cooperating microprocessors or the like, for example. For example, a cloud system or a database can come into consideration as a storage facility. The transfer of the program can thus also take place within a data network and/or the Internet.

The claims and the description below each contain particularly advantageous embodiments and developments of the invention. In particular the claims of one claim category can also be developed similarly to the claims of another claim category. In addition, in the context of the invention, the different features of different example embodiments and claims can also be combined to new example embodiments.

Preferably, the medical technology facility comprises an operating facility which is configured to be visualized on the display unit of the remote communication facility according to at least one embodiment of the invention and to be controlled by the remote communication facility.

Advantageously, an operation can be carried out remotely, for example, by a particularly competent person without the person needing to be on site. For example, in this way, life-saving operations or complex X-ray recordings can be carried out at inaccessible locations, for example, an Antarctic station, even if no suitable medical personnel are present on site.

The medical technology facility can also comprise an X-ray imaging facility, preferably in the form of an automatic X-ray imaging machine. Advantageously, a triggering of the X-ray radiation and a monitoring of the patient can take place from a distance that is safe for the operating personnel. The X-ray imaging facility can also be part of an operating facility and can serve to monitor a region to be treated with an operation in the interior of a patient.

Preferably, the display unit of the remote communication facility is configured to display a three-dimensional representation of the digital twin.

Advantageously, an operating person can observe the medical technology facility from different sides and possibly also remotely operate control panels arranged on different sides of the medical technology facility, which are displayed to the personnel in a digital form. Therefore, the operating person receives an overview of the medical technology facility which is possibly superior to observation from nearby since obstacles can simply be hidden in the digital representation by the display unit.

Particularly preferably, the remote communication facility according to the invention has a graphical representation environment with which the digital twin is rotatable so that a view of the digital twin from different sides is enabled. Such a graphical representation can also take place in the form of a virtual reality representation which, apart from a pictorial representation, has further perceptible components. Advantageously, the medical technology facility can be observed from any desired directions, so that an operating person can also select observation perspectives which are difficult to realize during direct observation from nearby.

The graphical representation environment is preferably configured such that individual objects which are situated in a region in which the medical technology facility is arranged can be hidden. Advantageously, visual obstacles can simply be hidden without having to be moved aside in reality.

Preferably also, the evaluating unit of the remote communication facility according to the invention is configured additionally to provide, apart from a graphical representation of the digital twin, additionally state data, for example, via a dashboard via the medical technology facility. This state data can comprise, for example, operational parameters and/or, generally expressed, functional data which instructs the operating person or another person using the remote communication facility about the current operational state of the medical technology facility. Advantageously, such a person preferably receives, in real time, a current feedback item regarding the state of the medical technology facility and the response behavior thereof to control actions of the operating person or influences from the proximity of the medical technology facility by other objects or persons.

Apart from an operating person of a medical technology facility, other persons can also communicate with the medical technology facility: for example, these are technicians as servicing personnel or members of the hospital leadership who exercise controller functions and a general overview of the use of the devices, the duration of use, perhaps patient types (weight, child/adult) or who wish to receive the statistics regarding the type of the interventions. This information indicates to the clinic management how well the device is utilized and possibly what device type (e.g. a device for adipose patients) will be needed in future. Real time demands generally do not exist with regard to such questions—typically, one update per day is sufficient.

If the medical technology facility is an imaging facility, then the state data comprises operational parameter data and machine data prepared for the respective utilization purpose.

The state data can preferably comprise one of the following data types:

the current device setting,

the organ program,

the battery level,

the time until the next service,

the operating hours,

accurate axis positions (for calculating the device pose),

possibly internal sensor values

and, if the imaging facility is an X-ray imaging facility, the X-ray tube voltage.

A current device setting comprises important operational parameters with which an examination or operation is carried out by a medical technology imaging facility. Knowledge of this information permits a targeted control action to be carried out.

An organ program makes available important parameter settings for the examination of individual organs in medical imaging.

Information regarding the charge state of a battery can inform about whether a medical examination can be carried out with a particular medical technology facility or not.

The time until the next service of a medical technology facility and the operating hours can be of interest for servicing personnel in order to set a date for the next servicing work. In addition, current sensor information in combination with machine learning algorithms can provide clues to an imminent defect. This information enables an exchange of the components before the actual failure.

The X-ray voltage influences the image quality during an X-ray recording and must be adapted, for example, to the type of recording and individual parameters of an examination person.

Particularly preferably, the display unit is configured so that a virtually simulated control panel of the medical technology facility can be represented. Advantageously, an operating person can remotely operate a control panel of a medical technology facility known to him, for example, via a touch screen function and does not have to readjust for the remote operation of a medical technology facility.

Preferably also, the remote communication facility according to the invention has an alternative control function based upon the digital twin. Such an alternative control function can enable, for example, an intuitive control possibility which realizes the selection of a target position in the virtual three-dimensional room displayed.

Advantageously, for example, a change in the position or orientation of an object, preferably the medical technology facility, can take place via a hand movement on the display unit or even a hand movement in the three-dimensional space.

It is particularly preferred that the control unit of the remote communication facility according to the invention is configured to control remotely a radiation release by an X-ray imaging facility. Advantageously, an operating person can protect himself against a radiation exposure from an X-ray device by starting the imaging remotely.

In some countries, the operating person does not enter the X-ray room even when the devices are switched off. Currently, the patient is positioned relative to the device with verbal instructions. During a use of a digital twin, the X-ray device can, for example, adapt its position to the human. The digital twin can support the operating person to position the patient correctly.

Preferably, the remote control facility according to the invention is configured, by modelling the environment and the persons and/or objects situated therein, to represent with the display unit a complex environment together with one or more medical technology facilities and to control them remotely. For capturing the complex environment, additional sensors, RFID tags for identifying persons, cameras, and sensors for position detection can be used. The pose of a person or an object can also be calculated by using artificial intelligence if parts of a person or an object are covered by obstacles. Advantageously, the digital twin can represent a whole operating theater or even a complex hospital environment by the integration of a plurality of facilities and the modeling of the environment and the persons active therein. Such an environment is highly dynamic due to the humans active within it. In order to include the persons in the digital twin, additional sensors can be installed for capturing the environment. Such sensors can comprise, for example, RFID tags for identifying the persons or cameras for capturing a current position and pose.

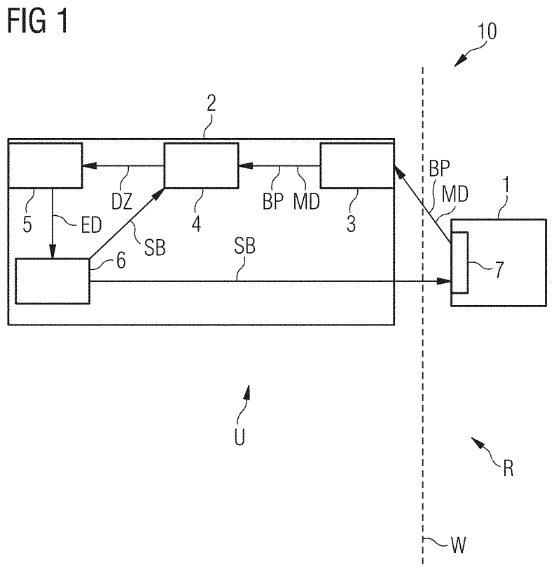

FIG. 1 is a schematic representation of a medical technology examination system 10 having an X-ray device 1 and a remote communication facility according to an example embodiment of the invention in the form of a remote operating facility 2. The X-ray device 1 is situated in an X-ray room R. In a room U which is separate from the X-ray room R, separated in FIG. 1 by a dashed line W, which indicates a greater spatial distance from the X-ray room R, is the remote control facility 2. The X-ray room R can be situated, for example, somewhere in a different building section than the remote control facility 2. The remote control facility 2 comprises a data capture unit 3 which receives state data comprising operational parameters BP and machine data MD from a control unit 7 of the X-ray device 1. The operational parameters BP and machine data MD are transferred to an evaluating unit 4 which generates a digital twin DZ based upon the operational parameters BP and the machine data MD and models of the X-ray device 1 and/or its environment. The digital twin DZ is displayed on a screen of a display unit 5 of an operating person (not shown). The operating person can now recognize a position and an operational state of the X-ray device 1 on the screen. If the operating person now wishes to carry out a control action, for example, an X-ray recording, he actuates a button and/or a knob, for example, on a control panel associated with the screen 5 in order to make an input. The input data ED is converted by a control unit 6 into control commands SB and transferred to the control unit 7 of the X-ray device 1. The control unit 7 then controls the X-ray device 1 based upon the control commands SB. For example, an imaging process is triggered which the operating person controls from the adjacent room. Firstly also, a control command SB can be sent to the evaluating unit 4 in order to illustrate an effect on the X-ray device 1 based upon the digital twin DZ. Firstly, based upon the control command, the digital twin DZ would be updated and/or generated by the evaluating unit 4 for a potential control action and transferred to the display unit 5 for display. Based upon this representation, the operating person can then decide whether the control command SB is to be carried out.

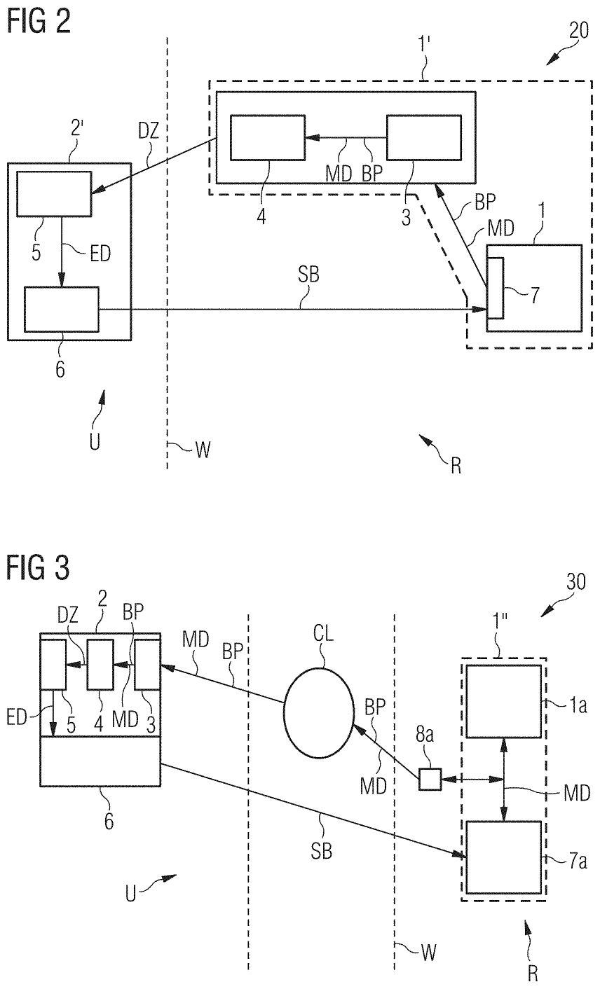

FIG. 2 shows schematically a medical technology examination system 20 having a remote communication facility 2′ according to a second example embodiment of the invention. The arrangement 20 shown in FIG. 2 differs from the arrangement shown in FIG. 1 in that the digital twin DZ is already generated in an X-ray imaging unit 1′. For this purpose, the data capture unit 3 and the evaluating unit are associated with an X-ray device 1 instead of—as with the arrangement 10 shown in FIG. 1—the remote communication facility 2. Therefore, a pre-processing of the data BP and MD of the X-ray device 1 already takes place in the X-ray room R and/or in the X-ray device 1′. In place of the operational parameter data BP and the machine data MD, data of a digital twin DZ is transferred to the cloud. The remote communication facility 2′ shown in FIG. 2 then receives the finished digital twin DZ and displays it for an operating person on a screen of a display unit 5. Through the transfer of input data ED, the operating person can then generate control commands SB on a control unit 6, which commands are transferred to the X-ray device 1 and/or the X-ray device 1′.

FIG. 3 shows schematically a medical technology examination system 30 according to a third example embodiment of the invention. The examination system 30 shown in FIG. 3 comprises an X-ray unit 1″ with an X-ray device 1a in an X-ray room R, the X-ray device being controlled by a computer unit 7a, also referred to as a control unit. The machine data MD generated by the computer unit 7a is transferred via a bus to the X-ray device 1a. A readout unit 8a is arranged on the internal bus system with which the bus is effectively “tapped” and the machine data MD is read out and converted into operational parameters BP. The operational parameters BP are transferred to a cloud CL and placed into intermediate storage there. The operational parameters BP and the machine data MD are transferred from the cloud CL to the remote control facility 2 which is constructed in the example embodiment of FIG. 3 exactly as shown in the example embodiment of FIG. 1. The remote control facility 2 receives the operational parameter data BP and the machine data from the cloud CL and, using an evaluating unit 4, determines a digital twin DZ based upon the operational parameter data BP. The digital twin DZ is displayed on a screen 5 for an operating person. Apart from the digital twin DZ, an operating panel is now also made available to the operating person at the usual location, which panel the operating person is able to actuate by touching knobs and/or buttons. If, for example, the operating person presses a button on the operating panel, then input data ED is generated in order, for example, to start an imaging process. For this purpose, corresponding control data and/or control commands SB are transferred from the control unit 6 to the control unit 7a of the X-ray unit 1″ for actuating the X-ray device 1a. The control unit 7a then starts the imaging by transferring corresponding machine data MD to the X-ray device 1a. The necessary machine data MD is already available on the internal bus of the X-ray unit 1″. The computer unit and/or readout unit 8a is also connected to this bus system and can therefore capture the important information.

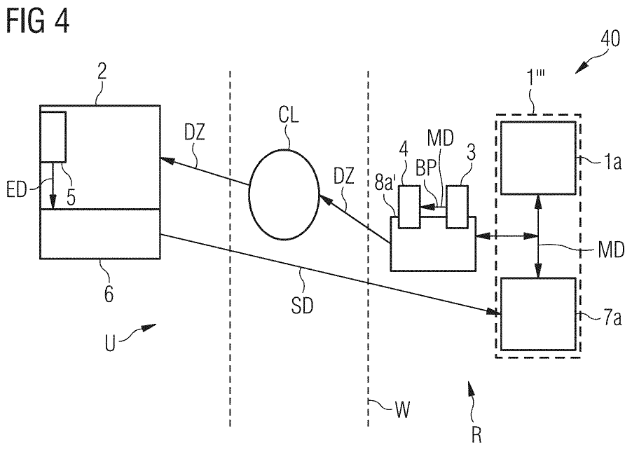

Depending upon the configuration, the raw data is then passed on by the readout unit 8a (see FIG. 3) or firstly the important information is calculated in the readout unit 8a via edge pre-processing (see FIG. 4).

On transfer to the cloud CL, the processed data is then transferred into a corresponding transfer protocol.

FIG. 4 shows schematically a medical technology examination system 40 according to a fourth example embodiment of the invention. The arrangement 40 shown in FIG. 4 differs from the arrangement 30 shown in FIG. 3 in that the digital twin DZ is already generated in a unit 8a′ associated with an X-ray imaging unit 1′″. For this purpose, the data capture unit 3 and the evaluating unit 4 are arranged in the readout unit 8a′ near the X-ray unit 1′″ rather than—as in the arrangement 30 shown in FIG. 3—as part of the remote control facility or remote communication facility 2′. Similarly to the X-ray unit 1″ shown in FIG. 3, the X-ray unit 1′″ comprises an X-ray device 1a and a control unit 7a for controlling the X-ray device 1a.

Therefore, a pre-processing of the data BP and MD of the X-ray device 1a or of the X-ray unit 1′″ comprising the X-ray device 1a already takes place in the X-ray room. In place of the operational parameter data BP and the machine data MD, data of a digital twin DZ is transferred to the cloud CL. The remote communication facility 2′ utilized in the fourth example embodiment corresponds to the structure according to the remote communication facility 2′ shown in FIG. 2.

FIG. 5 schematically shows a flow diagram 500 which schematically illustrates a remote communication method for remote control of a medical technology device according to an example embodiment of the invention. In step 5.I, firstly state data, for example, operational parameter data BP and machine data MD, is captured from a medical technology facility. Subsequently, in step 5.II, the captured state data BP, MD is used to generate a digital twin DZ. Then, in step 5.III, the digital twin DZ is displayed to an operating person in another room remote from the room of the X-ray device. Finally, in step 5.IV, a spatially remote controlling of the medical technology facility takes place based upon the displayed data of the digital twin by the operating person. For this purpose, the operating person carries out an operating action which is transferred to the medical technology device and/or to a control unit of the medical technology device and on the basis thereof, the medical technology device is remotely controlled.

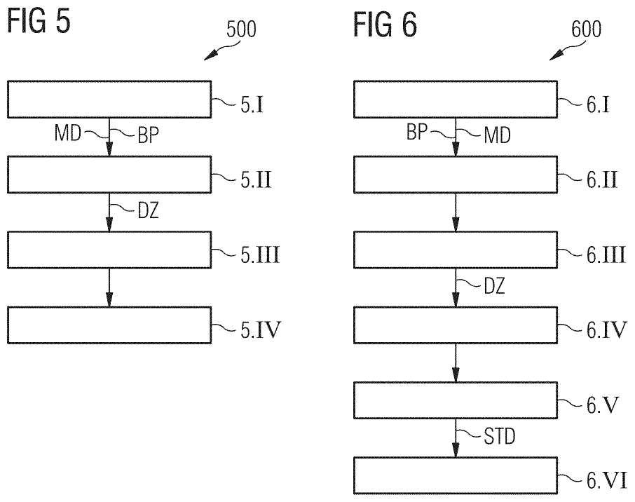

FIG. 6 shows a flow diagram 600 which illustrates the data transfer processes on a reproduction of a state of a medical technology device on a display of a remote communication facility. The flow diagram shown in FIG. 6 illustrates the data transfer already roughly outlined in relation to steps 5.I to 5.III. In step 6.I, machine data MD is generated which is used for controlling a medical technology device via a local control unit, for example, a computer unit, and operational parameters BP for an adjustment of a medical technology device are generated. In step 6.II, the machine data MD and operational parameters BP are registered on a bus interface. The machine data MD is subsequently evaluated in step 6.III by a computer unit and, based upon the machine data, a digital twin DZ is generated. The digital twin DZ is then transferred in step 6.IV to a cloud CL. In step 6.V, a transfer of the digital twin DZ as stream data STD to a client device with a suitable runtime and development environment, for example, Unity, takes place. Finally, in step 6.VI, with the aid of this runtime and development environment, a representation of a current pose and/or a current state of the medical technology device is carried out. Similarly to the example embodiments shown in FIGS. 1 to 4, a processing of the operational parameters BP and the machine data MD can be realized both on this side, i.e. in the control room and also on the other side, i.e. in the X-ray room. The variant illustrated in FIG. 6 represents the procedure in FIG. 4, which is also designated edge pre-processing. The processing of the captured raw data BP, MD, however, can also take place in the cloud and/or in the local client environment, i.e. in the region of the control room, as shown in FIG. 1 and FIG. 3. A combination (not shown) of the aforementioned procedures is also possible.

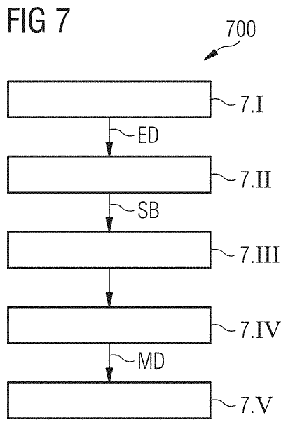

FIG. 7 shows a flow diagram which illustrates the data transfer processes on controlling a medical technology device via a remote control unit and/or a remote communication facility. The steps 7.I to 7.V are to be understood as a detailed illustration of the step 5.IV. In step 7.I, a control movement by an operating person on the usual operating panel or a computer of a remote communication facility according to one example embodiment of the invention takes place, wherein corresponding input data ED is generated. Subsequently, in step 7.II, the control movement and/or the input data ED thereby generated is converted into a control command SB. In step 7.III, the control command SB is transferred to a control unit associated with the medical technology device. In step 7.IV, the control unit generates a machine command and the machine command and/or the machine data MD generated for transferring the machine command is transferred in step 7.V to the medical technology device which carries out the generated control command, for example, to start an imaging process.

Finally, it should again be noted that the methods and apparatuses described above are merely preferred example embodiments of the invention and that the invention can also be modified by a person skilled in the art without departing from the field of the invention, to the extent that it is specified by the claims. Thus, the method and the remote communication facility have been described primarily based upon a system for recording medical image data. However, the invention is not restricted to the application described, rather the invention can, in principle, also be used for other purposes in the field of medicine. For the sake of completeness, it should also be mentioned that the use of the indefinite article “a” or “an” does not preclude the relevant features from also being present plurally. Similarly, the expression “unit” does not preclude this consisting of a plurality of components which can possibly also be spatially distributed.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A remote communication facility, comprising:
a data capture unit to capture state data from a medical technology facility;
an evaluating unit to generate a digital twin based upon the state data;
a display unit to display the digital twin; and
a communication unit to communicate with the medical technology facility based upon displayed data of the digital twin.

2. The remote communication facility of claim 1, wherein communication via the communication unit comprises one of:
controlling the medical technology facility;
monitoring the maintenance condition of the medical technology facility; and
monitoring a state of a patient located in a region of the medical technology facility.

3. The remote communication facility claim 1, wherein the display unit is configured to display a three-dimensional representation of the digital twin.

4. The remote communication facility of claim 1, having a representation environment for virtual reality with which the digital twin is rotatable so that a view of the digital twin from different sides is enabled.

5. The remote communication facility of claim 4, wherein the representation environment for virtual reality is configured such that individual objects, situated in a region in which the medical technology facility is arranged, are hideable.

6. The remote communication facility of claim 1, wherein the evaluating unit is configured to provide, apart from a graphical representation of the digital twin, additionally state data relating to the medical technology facility.

7. The remote communication facility of claim 1, wherein the state data includes operational parameters and machine data prepared for respective utilization purpose.

8. The remote communication facility of claim 1, wherein the display unit is configured to present a virtually recreated control panel of the medical technology facility.

9. The remote communication facility of claim 1, including an alternative control function based upon the digital twin.

10. The remote communication facility of claim 1, wherein the control unit is configured to remotely control a radiation release by an X-ray imaging facility.

11. The remote communication facility of claim 1, wherein the display unit is configured to represent, by modelling environment and at least one of persons and objects situated in the environment, a complex environment together with one or more medical technology facilities and wherein the remote communication facility is configured to control the complex environment together with one or more medical technology facilities remotely.

12. An operation and treatment system, comprising:
at least one of an operation and treatment room including at least one medical technology facility; and
a control room, spatially separated from the at least one of operation and treatment room, including the remote communication facility of claim 1.

13. A remote communication method, comprising:
capturing state data including at least one of machine data and operational parameters of a medical technology facility;
generating a digital twin based upon the state data;
spatially remotely displaying the digital twin; and
spatially remotely communicating with the medical technology facility based upon displayed data of the digital twin.

14. A non-transitory computer program product storing a computer program, directly loadable into a storage facility of a data processing facility, including program portions to carry out the method of claim 13 when the computer program is executed in the data processing facility.

15. A non-transitory computer-readable medium storing program portions, readable in and executable by a computer unit, to carry out the method of claim 13 when the program portions are executed by the computer unit.

16. The remote communication facility claim 2, wherein the display unit is configured to display a three-dimensional representation of the digital twin.

17. The remote communication facility of claim 2, having a representation environment for virtual reality with which the digital twin is rotatable so that a view of the digital twin from different sides is enabled.

18. The remote communication facility of claim 2, wherein the display unit is configured to present a virtually recreated control panel of the medical technology facility.

19. The remote communication facility of claim 2, including an alternative control function based upon the digital twin.

20. The remote communication facility of claim 2, wherein the control unit is configured to remotely control a radiation release by an X-ray imaging facility.
